(19) United States
(12) Patent Application Publication
(10) Pub. No.: US 2012/0226352 A1
(43) Pub. Date: Sep. 6, 2012

(54) SELF SUPPORTING AND FORMING BREAST IMPLANT AND METHOD FOR FORMING AND SUPPORTING AN IMPLANT IN A HUMAN BODY

(76) Inventor: Hilton Becker, Boca Raton, FL (US)

(21) Appl. No.: 13/462,294

(22) Filed: May 2, 2012

Related U.S. Application Data
(63) Continuation-in-part of application No. 12/766,821, filed on Apr. 23, 2010.
Application No. 12/766,821 is a continuation-in-part of application No. 12/556,050, filed on Sep. 9, 2009, now Pat. No. 8,197,542.
Application No. 12/556,050 is a continuation-in-part of application No. 12/552,353, filed on Sep. 2, 2009, now Pat. No. 8,202,317.

(51) Int. Cl. A61F 2/12; B29C 65/00

(52) U.S. Cl. 623/8; 29/428

(57) ABSTRACT

A breast implant includes an elastomer shell; a dermal matrix disposed on the implant shell; and a support interposed between the elastomer shell and dermal matrix to separate the elastomer shell and dermal matrix. A process for making a breast implant includes disposing an elastomer shell in a support; and disposing a dermal matrix on an outer surface of the support. A method of using the breast implant include disposing the breast implant in a subject and attaching the support to tissue of the subject.

### CROSS REFERENCE TO RELATED APPLICATIONS

This application is a continuation-in-part application of U.S. patent application Ser. No. 12/766,821, filed Apr. 23, 2010, which is a continuation-in-part application of U.S. patent application Ser. No. 12/556,050, filed Sep. 9, 2009, which is a continuation-in-part application of U.S. patent application Ser. No. 12/552,353, filed Sep. 2, 2009, the entire contents of which are incorporated herein by reference.

### FIELD OF THE INVENTION

This invention relates to a self forming and self supporting implant for breast reconstruction following a mastectomy, breast augmentation or the treatment of breast implant complications especially capsular contraction and more particularly to a method for forming and supporting a breast implant in a human body. The implant used is a tissue expander or an adjustable implant as disclosed in my earlier patents.

### BACKGROUND FOR THE INVENTION

Implants for breast augmentation and/or reconstruction are well known and have been in use for over 20 years. During that period, the implants have undergone a number of significant changes. For example, early implants had a smooth outer shell; however, as developments progressed the smooth shell was replaced with a textured surface. This was done in an effort to reduce problems associated with capsular contraction and to support a natural or pear shaped implant in position.

One approach to the use of a textured surface is disclosed in my co-pending U.S. patent application Ser. No. 12/169,000 filed on Jul. 8, 2008, that is a Continuation-In-Part of U.S. patent application Ser. No. 12/026,032 filed on Feb. 5, 2008. As disclosed therein, a method for texturing the surface of a synthetic implant includes the steps of providing an implant having a textured outer layer of silicone elastomer having a plurality of cavities filled with tissue growth enhancing material. Portions of the tissue growth enhancing materials protrude outwardly from the filled cavities. The implant also includes a hollow core filled with a fluid gel or liquid of silicone, saline or soy and a layer or mass of a biologically active non-absorbable material such as non-absorbable acellular dermis. The method also includes the step of forming a capsular pouch from the mass of biologically active non-absorbable material, placing the implant into the pouch and implanting the pouch containing the implant behind the breast thus holding the implant in position and reducing capsular contraction by the surrounding tissue and blood vessels growing into the acellular dermis. In a preferred embodiment of my earlier invention the acellular dermis and collagen are combined with hyaluronic acid and partially impregnated in the outer layer of a silicone elastomer so that the patient's blood vessels and tissue grow into the biologically active non-absorbable or only partially absorbable filled cavities to thereby anchor the implant in place.

Essentially an implant consists of a smooth silicon bag filled with either silicon gel or saline. When inserted into the body the implant is walled off by the response of the human tissue. This is commonly referred to as encapsulation. As the capsule that is formed is scar tissue, it is fairly rigid and in certain cases may actually contract resulting in hardening around the implant. This often requires further surgery with unpredictable results. The incidence of capsular contracture is approximately 20% and is even higher in patients undergoing mastectomy requiring breast reconstruction. Besides causing tightening around the implant, contracture leads to displacement, pain, distortion and discomfort. This reaction to the implant is known as a foreign body reaction. It is commonly seen in all biological tissue, as for example an oyster forming a pearl around a grain of sand. In the human, capsular contracture may become so severe that calcification actually occurs. The implant then becomes palpable and distorted.

In an effort to decrease capsular contracture it was believed that texturing the surface of the implant would disrupt the capsular tissue, thus resulting in less capsular contracture. For example, a U.S. Pat. No. 6,913,626 advocates covering the implant with a bio absorbable material in an attempt to reduce capsular contracture. Another U.S. Pat. No. 4,648,880 utilized a woven mesh is draped around the implant in an attempt to reduce capsular formation.

Texturing of the outer surface of the implant has not shown much success in decreasing capsular contracture, so much so that most surgeons have now reverted back to smooth implants. However, none of the prior art, which attempts to disrupt the collagen fibers in the capsule, have advocated creating a larger capsule which will form around the implant following which the implant can be reduced in volume which would then reduce the incidence of capsular contracture. None of the prior art suggests the use of a tissue expander combined with a mesh that would become integrated into the capsule at a larger volume and subsequently reduce the volume of the implant. This technique has been performed by the inventor on multiple surgical cases with excellent results. Thus, it has now been found that the most desirable implant is a smooth implant that results in a soft, natural feel. In summary, this technique is the use of a smooth implant that is left at a smaller volume than the matured mesh supported capsule.

Notwithstanding the above it is the Applicant's belief that there is a need and a potential market for an improved textured or smooth surface implant and a method for breast implant reconstruction and augmentation in accordance with the present invention. There should be a need and a market for such implants because they provide better anchoring, shape enhancement and fewer problems with capsular contraction. Further, it is believed that such implants can be marketed at a competitive price, are durable, improve the rate of healing and lead to more satisfactory results.

### BRIEF SUMMARY OF THE INVENTION

Disclosed in an embodiment is a breast implant comprising: an elastomer shell; a dermal matrix disposed on the implant shell; and a support interposed between the elastomer shell and dermal matrix to separate the elastomer shell and dermal matrix.

In another embodiment, a process for making a breast implant comprises: disposing an elastomer shell in a support; and disposing a dermal matrix on an outer surface of the support.

In a further embodiment, a method of using a breast implant comprises disposing the breast implant of claim 1 in a subject; and attaching the support to tissue of the subject.

The invention will now be described in connection with the following drawings wherein like numbers have been used to identify like parts.

### DESCRIPTION OF THE DRAWINGS

FIG. 1 is a schematic illustration of a generally cone shaped support;

FIG. 2 is a schematic illustration of an implant disposed in a mesh support in accordance with the present invention;

FIG. 3 is a flow chart illustrating a method for forming and supporting a breast implant in accordance with the present invention;

FIG. 4 is a cross-sectional view of a breast including a breast implant disposed therein in accordance with a preferred embodiment of the invention;

FIG. 5 is a schematic illustration of an implant with a mesh pouch enclosing the implant and including absorbable hooks in a rear portion thereof for attachment to a patient's tissue;

FIG. 6 is a schematic cross-sectional view of a breast with a dual lumen expandable mammary prosthesis implanted therein;

FIG. 7 is a schematic illustration of a breast implant in accordance with a preferred embodiment of the invention;

FIG. 8 is a schematic illustration of a smooth, inflatable implant with a textured mesh layer of partially absorbable layer thereon;

FIG. 9 is a schematic illustration of an expanded implant and textured mesh surface wherein the volume of the implant has been expanded by adding saline or the like that allows human tissue to grow into the mesh coating;

FIG. 10 is a schematic illustration of a smooth inflatable implant with a reduced volume to provide a space between the implant and the textured mesh layer and capsular surface;

FIG. 11 is an expanded portion of the breast and implant shown in FIG. 10;

FIG. 12 is cross-section of a breast implant having a dermal matrix disposed on an anterior portion of a support disposed on an elastomer shell;

FIG. 13 is a cross-section of a breast implant having a dermal matrix disposed on an inferior portion of an anterior surface of a support disposed on an elastomer shell;

FIG. 14 is a cross-section of a breast implant having a dermal matrix disposed on an inferior portion of a support disposed on an elastomer shell;

FIG. 15 is a perspective view of a breast implant having a dermal matrix disposed on a superior and inferior portion of a support;

FIG. 16 shows a breast implant disposed and attached to muscle tissue; and

FIG. 17 shows a breast implant interposed between muscle tissue.

### DETAILED DESCRIPTION

It has been found that a breast implant having an elastomeric shell disposed in a support that is at least partially covered with a dermal matrix provides decreased tissue irritation, ready bio-absorption of certain components of the breast implant, and mechanically strengthens or stabilizes compromised tissue. In addition the breast implant herein allows for rapid tissue in-growth in an outer portion of the implant and is strong enough to hold the implant and surrounding tissue in position after implantation of the breast implant. Further, the implant has a shape that is alterable and flexible in situ.

In essence a self supporting breast implant for breast augmentation and/or reconstruction comprises and/or consists of a generally cone shape support and an implant disposed within the support. In an embodiment, the support is formed from a partially absorbable, totally absorbable, non-absorbable material, or a combination comprising at least one of the foregoing. In a particular embodiment, the support is made from a sheet of ULTRAPRO® partially absorbable light weight surgical mesh consisting of about 75% polypropylene (non-absorbable) and 25% poliglecaprone (absorbable) monofilament materials, available from Ethicon Inc., a Johnson and Johnson company located in Langhorne, Pa.; a non-absorbable polytetrafluoroethylene (PTFE) mesh (e.g., infinite meshPTFE, available from Gore Co. in Flagstaff, Ariz.); a glycolide, lactide, and trimethylene carbonate surgical mesh (e.g., TIGR matrix surgical mesh available from Novus Scientific); totally absorbable material such as polyglactin (e.g., Vicryl available from Ethicon Corp., having total absorption within 6 months of implantation); or a combination comprising at least one of the foregoing. In practice, a disc shaped piece of mesh has a triangular piece removed and the edges left by the removal of the triangular piece are joined together to form a three dimensional cone shaped support. The implant comprises a silicone shell, a hollow core and a silicone or saline fluid dispersed in the hollow core.

The implant is placed within the cone and because of the flexibility of the mesh forms a generally pear or natural shape of a breast. The mesh also includes means such as an overlap of the mesh material for suturing to a patient's tissue or in the alternative including two or three absorbable hooks.

The invention also contemplates a method for forming and supporting a breast implant including the steps of providing a breast implant and a mass of partially or long term (e.g., greater than one year before absorption) absorbable light weight surgical mesh. The surgical mesh is formed into a generally cone shaped support and the implant, a smooth sided, non-absorbable shell is disposed in the cone shaped support. In a preferred embodiment of the invention the base of the cone is enclosed with a sheet of the mesh material so that the implant, preferably having a smooth surface, is fully covered by the mesh support that is in close proximity thereto and the mesh support or an extension thereof is surgically attached to the patient's tissue to thereby provide an internal bra-like support. Also, because of the flexibility of the mesh material, the cone shaped mesh in combination with the generally round or slightly pear shaped implant takes on the natural form of a breast. Ideally the mesh support pouch is used with an adjustable implant. With an adjustable gel implant, the implant can be expanded to further enhance the shape.

In a preferred embodiment of the present invention the implant comprises a tissue expander or an adjustable implant comprising an implant having an upper portion and a lower portion and wherein the implant lower portion is supported by a cup or sling shaped mesh support member supporting and surrounding a lower portion of said implant while leaving the upper portion thereof free of the support. The mesh support is adaptable to hold the implant in position with respect to a muscle underlying an incision without piercing the implant. An important aspect of this preferred embodiment resides in an expandable support that is elastic in all directions that can be expanded to enable a larger mesh supported capsule to be formed. This enables the surgeon to place a tissue expander or adjustable implant at the time of surgery, expand the implant, allow incorporation of the meshed outer layer and then reduce the volume thus resulting in a soft, pliable implant without surrounding tissue capsular contracture.

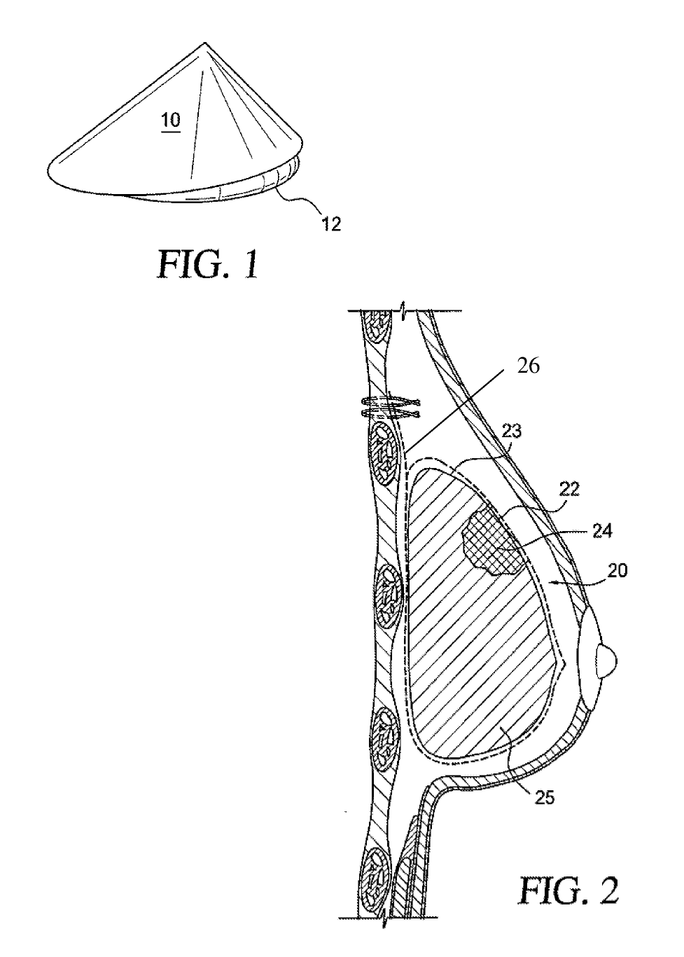

As illustrated in FIG. 1 a generally cone shaped support member 10 is formed from a mass or sheet of medical mesh as for example ULTRAPRO®, partially absorbable material from Ethicon Inc., a Johnson and Johnson company located in Langhorne, Pa., a totally absorbable material, non-absorbable material, or a combination comprising at least one of the foregoing. This mesh, which is used in an embodiment, is constructed of a combination of polypropylene (non-absorbable) and poliglecaprone, (absorbable), monofilament material in varying percentages, i.e., 80% absorbable and 20% permanent. In another embodiment, the mesh is polypropylene (non-absorbable) and poliglecaprone, (absorbable), monofilament material in varying percentages, i.e., 80% absorbable and 20% permanent; TIGR matrix surgical mesh; a long term absorbable material; Vicryl; or a combination comprising at least one of the foregoing. Thus, the support member 10 can be partially absorbed, totally absorbed, or non-absorbed after implantation.

The absorbable portion of the mesh may encompass the whole surface, or part of the surface, i.e., the upper portion can be predominantly absorbable while the lower part is predominantly non absorbable. This will allow a sling of support to remain while the upper portion is absorbed. The direction of the elasticity can also be configured so as to enhance the shape of a round implant on expansion, i.e., if the anterior lower portion is made more elastic than the upper portion, so that a round implant will assume a pear shape.

It is contemplated that other synthetic meshes may be used as for example polypropylene mesh with filament diameters ranging from 0.08 mm to 0.20 mm, pore size from about 0.8 mm to 3.0 mm, and finer, and weights from 25 grams per square meter (g/m²) to 100 g/m². Other materials include polyester felt, polyester knitted mesh, polytetrafluoroethylene, nylon, etc. In addition, various other types of mesh may be used in forming a support. For example, biological mesh made from collagen sheets of human and animal origin, synthetic woven mesh as for example nylon, polyethylene terephthalate (available under the name Dacron), Gore-Tex (e.g., thermo-mechanically expanded polytetrafluoroethylene (PTFE) with other fluoropolymer products), and combination meshes with strands of nylon interwoven with strands of collagen.

The generally cone shaped support 10 may be formed by taking a disc shaped piece of mesh and removing a triangular portion from the disc. The edges of the pie shape with a piece removed are then joined together to form a generally three dimensional cone shape as shown in FIG. 1. As shown, the cone shaped member 10 is placed on top of a silicone shell 12 that is filled with a silicone or saline fluid. In a preferred embodiment of the invention, the implant is partially enclosed in a bag or sling like support that includes the cone shaped structure.

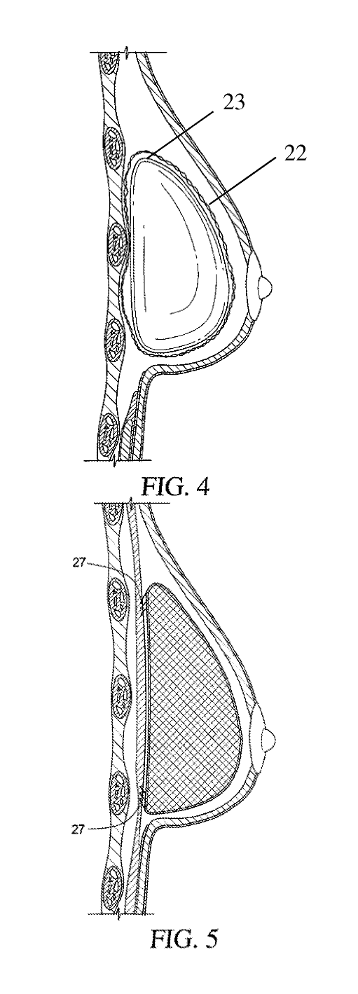

As shown in FIG. 2 an improved breast implant 20 in accordance with one embodiment of the invention includes a mesh pouch 22 of a light weight partially absorbable monofilament material and a non-absorbable silicone shell 23 (also referred herein as an implant shell or elastomer shell) that defines a hollow core 24 that is filled with silicone gel 25 or the like. The implant also includes an extension 26 of the mesh pouch 22 that is sutured to a patient's tissue. The implant 23 includes a tube (not shown) leading to the pouch 22, but held within the surgeon's hand. A remote port and tube are of conventional design and typically used to insert additional saline or silicone fluid or reduce the fluid filler from the inner prosthesis. The inner non-absorbable prosthesis or implant 23 is shown in FIGS. 4 and 5 wherein the inner prosthesis or inner implant 23 is encased in the mesh pouch 22. The mesh pouch is closed and has an upward portion 26 to provide a fully enclosed structure. The upward portion 26 can be a strip of mesh (e.g., the same material as mesh pouch 22) that extends outwardly to enable sutures to be placed on the mesh for anchoring to body tissue.

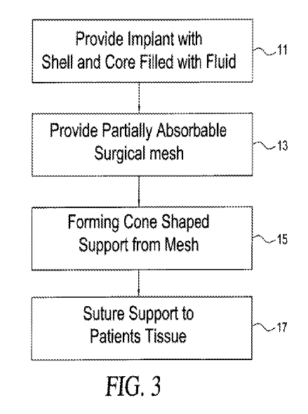

As illustrated in FIG. 3 a method in accordance with the present invention contemplates the following steps. The method includes the step 11 of providing an implant with an outer shell of medical grade silicone or the like having an inner core that is filled with a silicone gel, saline, etc. The method also includes the step 13 of providing a partially absorbable surgical mesh that is formed into a cone-shaped support in step 15 that is subsequently attached to a patient's tissue in step 17.

In FIG. 4, the pouch 22 is slightly spaced from the outer shell 23 in a manner that appears to minimize problems associated with capsular contraction.

It is also contemplated that rather than stitching the implant to the patient's tissue, the implant may be readily attached by a plurality of absorbable hooks 27 as shown in FIG. 5.

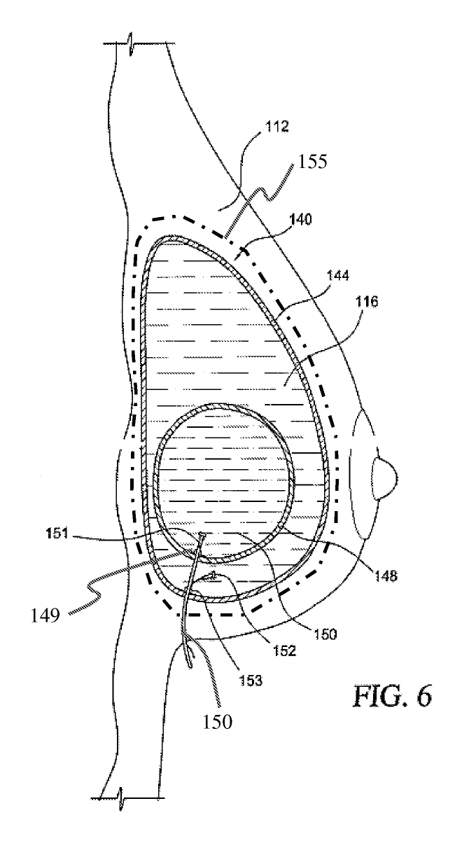

In a further embodiment shown in FIG. 6, a breast implant 112 has an outer elastic shell 144 and an inner elastic shell 148. A space 116 between the inner elastic shell 148 and the outer elastic shell 144 can be filled with a fluid, e.g., saline or silicone gel. The space 150 inside inner shell 148 also can be filled with a fluid, e.g., saline or silicone gel, independently of outer elastic shell 144. Filling tube 150 interfaced to valve 149 and valve plug 151 controls the fill volume of the inner elastic shell 148 as well as isolate the inner elastic shell 148 from the outer elastic shell 144 to prevent leakage of the inner elastic shell 148. Similarly, a valve 153 and plug 152 respectively connected to the outer elastic shell 144 and the filling tube 151 isolate the outer elastic shell 144 from leaking once filling tube 151 is removed. A mesh pouch 155 is surroundingly disposed over the outer elastic shell 144.

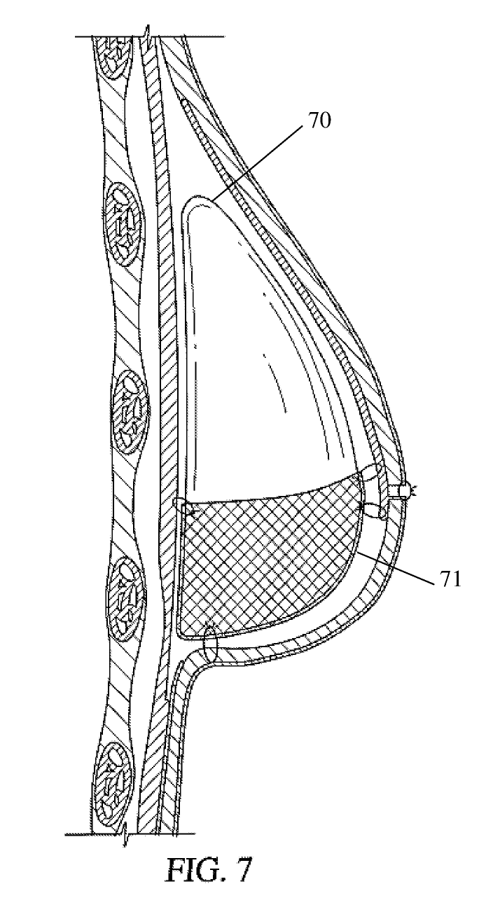

A preferred embodiment of the invention is illustrated in FIG. 7 wherein a self supporting breast implant comprises an adjustable implant 70 having an upper and a lower portion and a generally cup or sling shaped mesh support 71 surrounding and supporting said lower portion of said implant 70 while leaving said upper portion thereof free of said mesh support 71 and wherein said mesh support 71 is adapted to hold the implant 70 in position with respect to a muscle underlying an incision without piercing the implant 70. Further, the implant 70 is susceptible to limited expansion in all directions and wherein the implant 70 has a smooth surface, is expanded and subsequently reduced in volume and preferably is partially absorbable in the human body. For example, the mesh sling 71 comprises a lightweight partially absorbable monofilament material that is made of silicone and filled with a mass of silicone, saline, or gel. In the most preferred embodiment of the invention the mesh support 71 comprises about 25-75 wt. % polypropylene and about 75-25 wt. % poliglecaprone monofilament, based on the total weight of the mesh support 71. The non-absorbable portion may be even of a lower percentage.

The invention also contemplates a method for breast augmentation by implanting a self-supporting adjustable breast implant in a patient. The method includes the steps of providing an implant having an upper and a lower portion with a generally cup or sling shape support surrounding and supporting the lower portion of the implant by leaving the upper portion thereof free of the mesh support and wherein the implant is positioned with respect to a muscle underlying an incision without piercing the implant.

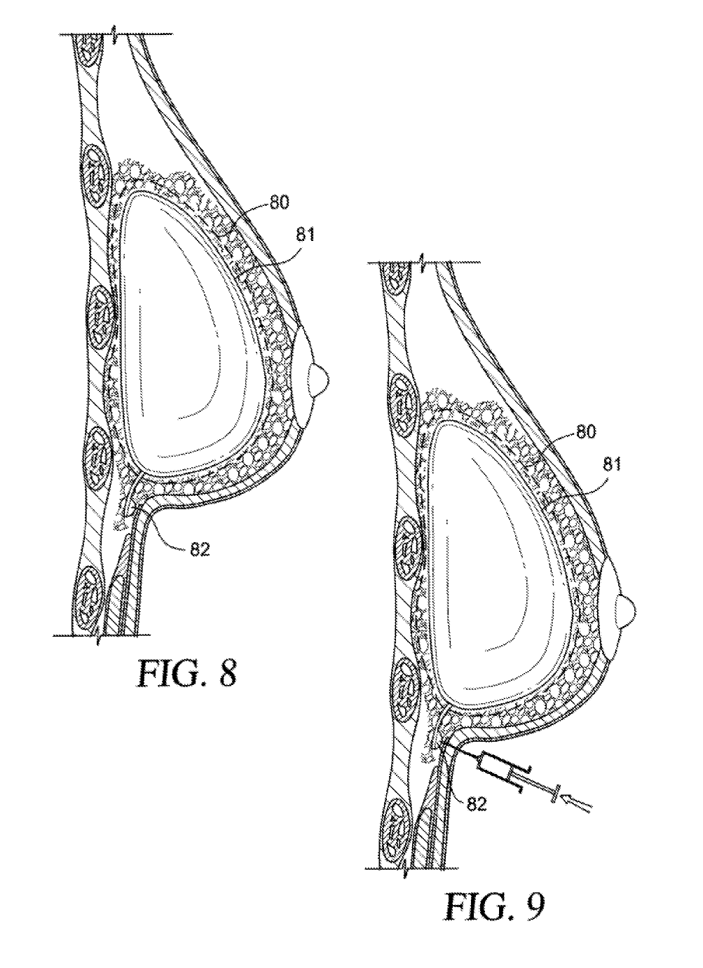

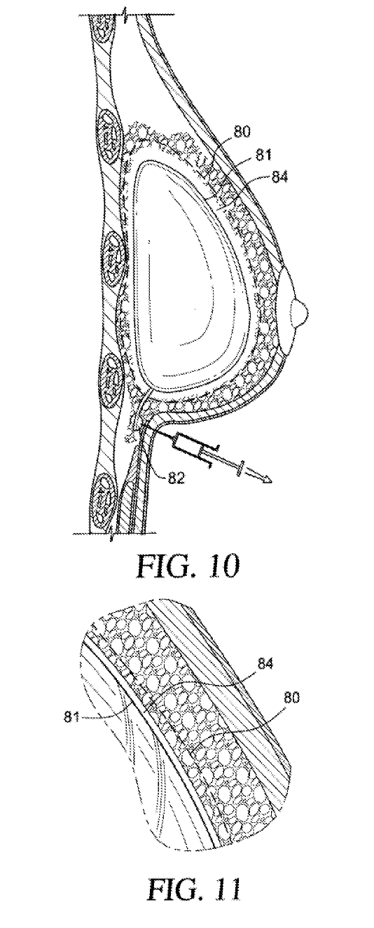

An implant as shown in FIG. 8 includes a mesh envelope 80 surrounding the shell of the implant 81 as implanted in a human breast. The implant or shell 81 is then expanded as shown in FIG. 9 by injecting saline or the like into the implant or shell 81 by means of an injection port 82. This injection increases the volume of the implant 81 and forces the mesh 80 into the surrounding tissue. After the mesh is adhered to the human tissue, the volume of the implant 81 is reduced by withdrawing fluid from or through the injection port 82. This leaves a space 84 between the implant 81 and a capsular tissue surface as shown in FIGS. 10 and 11.

The invention further contemplates coating the non-absorbable mesh or impregnating the absorbable mesh with an antibiotic. The antibiotic can be, for example, an aminoglycoside, quinolone, or β-lactam. Exemplary antibiotics include ciprofloxacin, gentamycin, tobramycin, erythromycin, vancomycin, oxacillin, cloxacillin, methicillin, lincomycin, ampicillin, colisin, cephalosporines, and the like such as antibiotics used in clinical use and in surgeries. In a non-limiting embodiment, an anti-inflammatory drug can be added to the mesh. In a further embodiment, the anti-inflammatory drug, e.g., particles thereof, can be included in the dermal matrix.

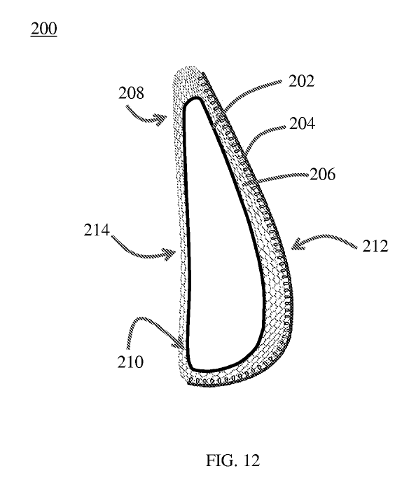

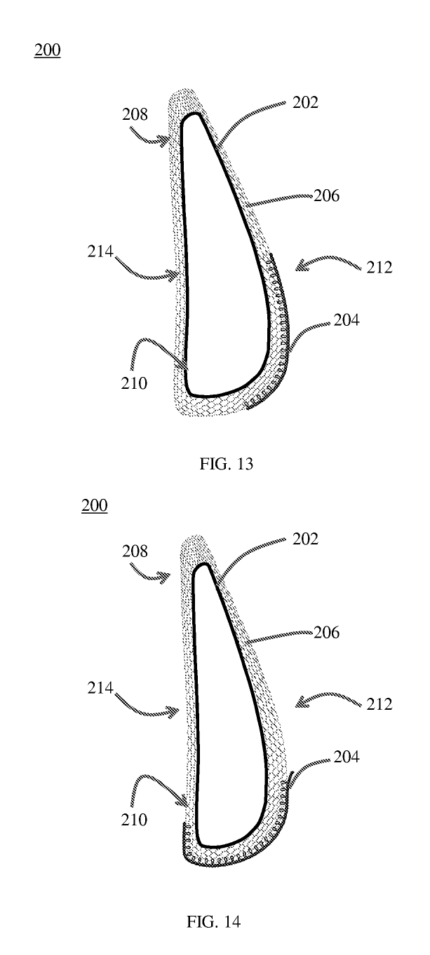

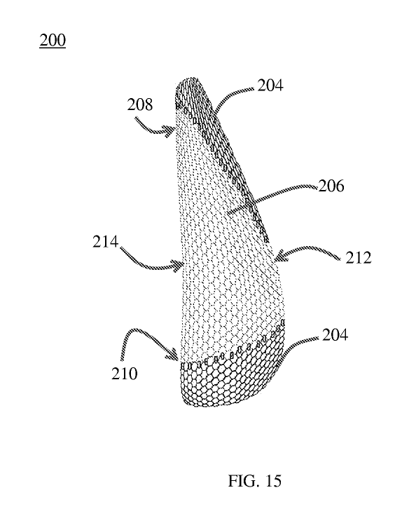

As shown in FIG. 12, according to another embodiment, a breast implant 200 includes an elastomer shell 202, a dermal matrix 204 disposed on the elastomer shell 202, and a support 206 interposed between the elastomer shell 202 and dermal matrix 204 to separate the elastomer shell 202 and dermal matrix 204. The breast implant 200 has a superior portion 208 and inferior portion 210. The support can be configured to receive attachment material to attach the breast implant to human tissue. Although many types of supports can be used, in an embodiment, the support 206 comprises a mesh enclosure. In some embodiments, the elastomer shell 202 is disposed completely in the support 206. In other embodiments, the elastomer shell 202 can be disposed partially in the support 206. Likewise, the dermal matrix 204 can be disposed partially on the support 206. In a further embodiment, the dermal matrix 204 is disposed only on the anterior surface 212 of the support 206, as in FIG. 12. Alternatively, the dermal matrix 204 can be disposed only on the anterior 212 and posterior surfaces 214 of the inferior portion 210 of the support 206 but not on the superior portion 208 of the support 206, as in FIG. 13. In a further embodiment, the dermal matrix 204 can be disposed on the inferior portion 210 of the anterior 212 and posterior 214 surfaces, as in the hammock-shaped dermal matrix shown in FIG. 14. In yet another embodiment, the dermal matrix 204 can be partially disposed on the superior 208, inferior 210, anterior 212, and posterior 214 portions of the support 206, as in the perspective view of the breast implant 200 shown in FIG. 15.

According to an embodiment, the dermal matrix includes biological material, synthetic material, or a combination comprising at least one of the foregoing. An exemplary dermal matrix includes an acellular dermal matrix, collagen, protein, amino acid, carbohydrate, polyethylene terephthalate, polycarbonate, polylactic glycolic acid, glycolide, lactide, trimethylene carbonate, or a combination comprising at least one of the foregoing. The support can include non-absorbable material, absorbable material, or a combination comprising at least one of the foregoing. Exemplary supports include a polypropylene, poliglecaprone, polylactide, polyglycolide, polydioxanone, polyglactin, caprolactone, glycolide, lactide, trimethylene carbonate, polyorthoester, polyethylene glycol, poly terephthalate, tyrosine, poly(ester amide), or a combination comprising at least one of the foregoing. In an embodiment, the elastomer shell comprises a silicone, polyisobutylene, poly(ethylene terephthalate), poly(tetrafluoroethylene), polypropylene, polyurethane, polystyrene, or a combination comprising at least one of the foregoing.

According to an embodiment, the breast implant further includes a hollow core (not shown be similar to the inner elastic shell 148 of FIG. 6) disposed in the elastomer shell 202, and a fluid disposed in the hollow core. Such fluid can include saline, silicone gel, polyvinyl pyrrolidone, hyaluronic acid, polyacrylamides, polysaccharides, dextran, methylcellulose-hydrogel, triglycerides, cellulose, or a combination comprising at least one of the foregoing.

Also contemplated is a process for making a breast implant that includes disposing an elastomer shell in a support and disposing a dermal matrix on an outer surface of the support. The dermal matrix can be affixedly connected to the support. Such connectivity can include chemical or mechanical fixing. Chemical fixing includes polymerization or other reactivity or bonding, including electrostatics. Mechanical fixing includes, e.g., coupling with a connector such as suture material.

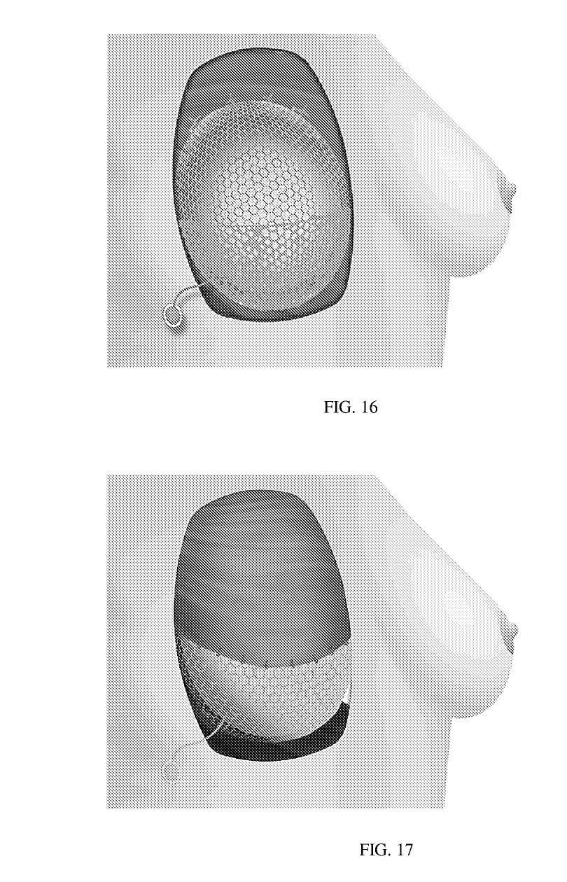

In an embodiment, a method of using a breast implant includes disposing the breast implant herein and attaching the support to tissue of the subject. This method further includes adjusting a volume of fluid disposed in the elastomer shell. In some embodiments, the volume is adjusted before, after, during an implantation surgery, or a combination of at least one of the foregoing periods. In some embodiments, the breast implant can be interposed between a pectoralis muscle and breast tissue of the subject, as in FIG. 16 (where the breast implant is shown covering the pectoralis muscle). In a further embodiment, the breast implant can be disposed under a pectoralis muscle of the subject, as in FIG. 17.

Once implanted, the support of the implant can totally or partially resolve, leaving the dermal matrix and elastic shell in position. Since various materials having different absorption rates are used for the support, the implant can be implanted in view of a time duration for which the support should remain in tact and interposed between the elastic shell and the dermal matrix.

While one or more embodiments have been shown and described, modifications and substitutions may be made thereto without departing from the spirit and scope of the invention. Accordingly, it is to be understood that the present invention has been described by way of illustrations and not limitation. Embodiments herein are can be used independently or can be combined.

All ranges disclosed herein are inclusive of the endpoints, and the endpoints are independently combinable with each other. The suffix “(s)” as used herein is intended to include both the singular and the plural of the term that it modifies, thereby including at least one of that term (e.g., the colorant(s) includes at least one colorants). “Optional” or “optionally” means that the subsequently described event or circumstance can or cannot occur, and that the description includes instances where the event occurs and instances where it does not. As used herein, “combination” is inclusive of blends, mixtures, alloys, reaction products, and the like. All references are incorporated herein by reference.

The use of the terms “a” and “an” and “the” and similar referents in the context of describing the invention (especially in the context of the following claims) are to be construed to cover both the singular and the plural, unless otherwise indicated herein or clearly contradicted by context. As used herein, the term “a” includes at least one of an element that “a” precedes, for example, “a device” includes “at least one device.” “Or” means “and/or.” Further, it should further be noted that the terms “first,” “second,” and the like herein do not denote any order, quantity (such that more than one, two, or more than two of an element can be present), or importance, but rather are used to distinguish one element from another. The modifier “about” used in connection with a quantity is inclusive of the stated value and has the meaning dictated by the context (e.g., it includes the degree of error associated with measurement of the particular quantity).

What is claimed is:

1. A breast implant comprising:
an elastomer shell;
a dermal matrix disposed on the implant shell; and
a support interposed between the elastomer shell and dermal matrix to separate the elastomer shell and dermal matrix.

2. The breast implant of claim 1, wherein the support is configured to receive attachment material to attach the breast implant to human tissue.

3. The breast implant of claim 1, wherein the support comprises a mesh enclosure.

4. The breast implant of claim 3, wherein the elastomer shell is disposed completely in the support.

5. The breast implant of claim 3, wherein the elastomer shell is disposed partially in the support.

6. The breast implant of claim 3, wherein the dermal matrix is disposed partially on the support.

7. The breast implant of claim 6, wherein the dermal matrix is disposed only on the anterior surface of the support.

8. The breast implant of claim 6, wherein the dermal matrix is disposed only on the anterior and posterior surfaces of the inferior portion of the support but not on the superior portion of the support.

9. The breast implant of claim 1, wherein the dermal matrix comprises biological material, synthetic material, or a combination comprising at least one of the foregoing.

10. The breast implant of claim 1, wherein the dermal matrix comprises an acellular dermal matrix, collagen, protein, amino acid, carbohydrate, polyethylene terephthalate, polycarbonate, polylactic glycolic acid, glycolide, lactide, trimethylene carbonate, or a combination comprising at least one of the foregoing.

11. The breast implant of claim 1, wherein the support comprises non-absorbable material, absorbable material, or a combination comprising at least one of the foregoing.

12. The breast implant of claim 1, wherein the support comprises a polypropylene, poliglecaprone, polylactide, polyglycolide, polydioxanone, polyglactin, caprolactone, polyglactin, trimethylene carbonate, polyorthoester, polyethylene glycol, poly terephthalate, tyrosine, poly(ester amide), or a combination comprising at least one of the foregoing.

13. The breast implant of claim 1, wherein the elastomer shell comprises a silicone, polyisobutylene, poly(ethylene terephthalate), poly(tetrafluoroethylene), polypropylene, polyurethane, polystyrene, or a combination comprising at least one of the foregoing.

14. The breast implant of claim 11, further comprising:
a hollow core disposed in the implant shell; and
a fluid disposed in the hollow core.

15. The breast implant of claim 14, wherein the fluid comprises saline, silicone gel, polyvinyl pyrrolidone, hyaluronic acid, polyacrylamides, polysaccharides, dextran, methylcellulose-hydrogel, triglycerides, cellulose, or a combination comprising at least one of the foregoing.

16. A process for making a breast implant, the method comprising:
disposing an elastomer shell in a support; and
disposing a dermal matrix on an outer surface of the support.

17. The process of claim 16, wherein the dermal matrix is affixedly connected to the support.

18. A method of using a breast implant, the method comprising:
disposing the breast implant of claim 1 in a subject; and
attaching the support to tissue of the subject.

19. The method of claim 18, further comprising adjusting a volume of fluid disposed in the elastomer shell.

20. The method of claim 19, wherein adjusting the volume is performed after an implantation surgery.

21. The method of claim 18, wherein the breast implant is disposed under a pectoralis muscle of the subject.
